(19) United States
(12) Patent Application Publication
(10) Pub. No.: US 2024/0002952 A1
(43) Pub. Date: Jan. 4, 2024

(54) METHOD FOR IN VITRO DIAGNOSIS OF MUCOSITIS IN CANCER PATIENTS UNDERGOING ANTITUMOR THERAPY

(71) Applicant: ISTITUTI FISIOTERAPICI OSPITALIERI, ROMA (IT)

(72) Inventors: Sabrina STRANO, Roma (IT); Aldo MORRONE, Roma (IT); Claudio PULITO, Roma (IT); Giovanni BLANDINO, Roma (IT); Andrea SACCONI, Roma (IT)

(21) Appl. No.: 18/267,743

(22) PCT Filed: Dec. 14, 2021
(86) PCT No.: PCT/IT2021/050407

§ 371 Date: Jun. 15, 2023

(30) Foreign Application Priority Data
Dec. 15, 2020 (IT) 102020000030833

(51) Int. Cl. C12Q 1/6886

(52) U.S. Cl. CPC C12Q 1/6886; C12Q 2600/118; C12Q 2600/178; C12Q 2600/158

(57) ABSTRACT

A method is for in vitro diagnosis of mucositis, especially, early-stage mucositis, in cancer patients undergoing antitumor therapy, through the Measurement of microRNA expression. The diagnosed cancer patients can be treated by administering a treatment indicated in guidelines provided by Multinational Association of Supportive Care in Cancer and International Society for Oral Oncology (MASCC/ISOO), by increasing interval time before a subsequent antitumor therapy is administered, or by varying dosage of the antitumor therapy.

### PRIORITY AND CROSS REFERENCE TO RELATED APPLICATIONS

This application is the U.S. National Phase Application under 35 U.S.C. § 371 of International Application No. PCT/IT2021/050407, filed Dec. 14, 2021, and published as WO2022/130433 on Jun. 23, 2022, which claims the benefit of Foreign Application No. IT102020000030833, filed Dec. 15, 2020. Any and all applications for which a foreign or a domestic priority is claimed is/are identified in the Application Data Sheet filed herewith and is/are hereby incorporated by reference in their entireties under 37 C.F.R. § 1.57.

### FIELD

The present invention relates to a method for in vitro diagnosis of mucositis in cancer patients undergoing antitumour therapy. In particular, the present invention concerns a method for in vitro diagnosis di mucositis, or rather, early-stage mucositis, in cancer patients undergoing antitumour therapy, through the measurement of microRNA expression in liquid biopsy samples.

### BACKGROUND

The term “oral mucositis” means an inflammatory state affecting the mucous membranes lining the oral cavity and the oropharynx, characterised by erythema, oedema, tissue atrophy and, in more advanced stages, ulcer.

It has been demonstrated that about 40% of patients who undergo chemotherapy develop mucositis; the percentage increases considerably (75-80%) in patients undergoing treatment with high chemotherapy doses [1]. Furthermore, mucositis is diagnosed in nearly all patients affected by head and neck cancer (HNC) who undergo a combined chemoradiotherapy treatment.

Mucositis represents a painful and debilitating condition, which may compromise oral food intake and consequently compromise the patient's quality of life. In the most advanced stages, it is necessary to hospitalise the patient for clinical management of the problem and interrupt the cancer treatment. It is estimated that about 16% of patients who undergo radiochemotherapy for the treatment of head and neck cancer are hospitalised for the management of mucositis [2]. Of these, 11% are forced to interrupt the cancer treatment [2].

In general, about 40% of patients affected by solid tumours develop treatment-induced mucositis. This phenomenon is also common in patients treated for lymphoma and leukaemia, as in the case, for example, of mucositis that occurs in young patients affected by acute lymphoblastic leukaemia who undergo a treatment with methotrexate, daunorubicin or cyclophosphamide.

Alongside the by now known etiologic role of chemotherapeutics in the pathogenesis of mucositis, in recent years the role of new molecularly targeted drugs in inducing the process have also begun to be highlighted. In particular, it has been observed that the evolution of mucositis varies based on the molecular agent used for the treatment. Mucositis associated with treatment with mTOR inhibitors has, in fact, peculiar clinical features differentiating it from radiotherapy- or chemotherapy-induced mucositis.

In addition to the painful and debilitating conditions that mucositis causes in a patient, managing cancer patients affected by mucositis also has a considerable impact in terms of costs. The concomitant presence of this pathology, a phenomenon that involves a wide range of HNC patients, in fact causes the hospitahsation of 16% of them, with a consequent increase in management costs, which strains the resources of the national health system. One study has shown that the cost of a patient who is hospitalised for treatment of a solid tumour or lymphoma is about 3893 dollars per cycle of therapy, whereas the cost arrives at about 6277 dollars if the patient has mucositis [3]. In particular, the cost for the health care system of a patient with head and neck cancer who undergoes chemoradiotherapy increases by 1700-6000 dollars, based on the degree of oral mucositis that develops [4].

There are currently no specific therapies for the treatment of mucositis; the current treatments, in fact, are only a support aimed at attenuating the symptoms. Recommendations have been defined for the prevention of mucositis; however, their effectiveness is rather low. As far as diagnosis is concerned, it is presently performed following the same guidelines used to measure the degree of mucositis, enabling the diagnosis thereof only in the presence of manifest disease. At present there exist various scales for measuring mucositis; consequently, the degree assigned varies according to the criteria established in the different scales. The World Health Organisation (WHO) uses, for example, objective criteria such as the presence or absence of ulcers, as well as the dimensions thereof in order to define the degree. A quantitative scale is instead used to measure mucositis as defined by the Oral Mucositis Assessment Scales COMAS). The ECOG Group (Eastern Cooperative Oncology Group) and the American National Cancer Institute (NCI) have included criteria for measuring the degree of mucositis among the general toxicity criteria common to chemotherapeutics, classifying the event mainly on the basis of the anatomic site in which it manifests itself.

The pathogenesis of mucositis is the result of a pathophysiological process triggered by the damage induced by antineoplastic treatment. In particular, the pathogenesis of mucositis is characterised by five stages divided into: initiation of tissue damage, signal transmission, signal amplification, ulceration/inflammation and repair [5]. In the first phase, the damaged tissue releases mediators that modulate the subsequent phases of inflammation and ulceration. In this context it would be useful to identify early markers of the response to the damage which can assure a timely management of the symptoms due to the disease. The studies conducted to date have measured the levels, in patients' saliva or serum, of circulating proteins such as growth factors, cytokines and interleukins. Furthermore, meta-analysis data, obtained by reviewing studies conducted for the detection of biomarkers predictive of the onset of oral mucositis in HNC patients, have shown that only 3 of the 27 factors taken into consideration are associated with a greater risk of developing radiotherapy-associated mucositis (epidermal growth factor, C-reactive protein and erythrocyte sedimentation rate). At the same time, the levels of none of the 27 seem to correlate with the degree of development of the mucositis itself [6].

In the light of the foregoing, there appears to be an evident need to provide new diagnostic methods capable of early detecting mucositis, and which overcome the disadvantages of the known methods.

The solution according to the present invention fits into this context; it aims to provide a new method of in vitro diagnosis that provides for the use of circulating miRNAs as predictive biomarkers for early diagnosis of the development of mucositis in patients undergoing antitumour therapies, such as, for example, in HNSCC patients.

MicroRNAs are a group of small non-coding RNAs (about 20 nucleotides) involved in the posttranscriptional regulation of messenger RNAs. By virtue of their particular ability to bind complementary sites present in the messenger RNA 3′ untranslated region, they play a key role in the regulation of all physiological processes. Their deregulation is in fact associated with the development of various diseases, including head and neck cancer (HNC) [7].

MicroRNAs are very stable molecules easily detectable in tissues fixed with formalin, preserved in paraffin and within the different biological fluids tested up to today. The analysis of their expression within tumour tissues and the associated biological fluids has shown that their release occurs in a tissue-specific manner. This characteristic, combined with their protection against the activity of endogenous RNase [8, 9], reflects their potential development as extracellular biomarkers [8, 10]. A variety of evidence has suggested the possibility of using microRNAs as biomarkers, by examining their expression in the saliva and serum of HNSCC patients [11, 12].

### SUMMARY

In some embodiments, a method for in vitro diagnosis and treatment of mucositis in a cancer patient undergoing antitumor therapy, includes obtaining a measurement, in at least one biological sample of liquid biopsy taken before the antitumor therapy and in at least one biological sample of liquid biopsy taken after the antitumor therapy, the expression of an miRNAs selected from the group consisting of hsa-miR-18b-3p, hsa-miR-494-3p, hsa-miR-1202, hsa-miR-3135b, hsa-miR-6769b-5p and/or hsa-miR-6875-5p; identifying a change in the expression of said miRNAs in at least one biological sample taken after the antitumor therapy compared to the expression of said miRNAs in at least one biological sample taken before the antitumor therapy where a change indicates mucositis; and carrying out at least (a) administering a treatment indicated in guidelines provided by Multinational Association of Supportive Care in Cancer and International Society for Oral Oncology (MASCC/ISOO); (b) increasing interval time before a subsequent antitumor therapy is administered; or (c) varying dosage of the antitumor therapy. In some embodiments, the mucositis is at a pathogenetic stage of initiation of tissue damage. In some embodiments, one, more than one or all of the miRNAs includes of hsa-miR-18b-3p. In some embodiments, a step of measuring the expression of one, more than one or all said miRNAs is carried out only in at least one biological sample taken after the antitumour therapy, when the one, more than one or all of the miRNAs consist of hsa-miR-18b-3p. In some embodiments, the expression of more than one of the miRNAs is measured and the miRNAs are selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-5p; hsa-miR-18b-3p and hsa-miR-6875-5p; hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b; hsa-miR-18b-3p, hsa-miR-1202 and hsa-miR-6769b-hsa-miR-18b-3p, hsa-miR-1202 and hsa-miR-6875-5p; hsa-miR-18b-3p, hsa-miR-3135b and hsa-miR-6769b-5p; hsa-miR-18b-3p, hsa-miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; miR-494-3p; hsa-miR-1202 and hsa-miR-6769b-5p; miR-494-3p, hsa-miR-1202 and hsa-miR-6875-5p; miR-494-3p, hsa-miR-3135b and hsa-miR-6769b-miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, miR-3135b and hsa-miR-6769b-5p; hsa-miR-18b-3p, miR-494-3p, miR-3135b and hsa-miR-6875-5p; hsa-miR-18b-3p; miR-494-3p, hsa-miR-6769b-5p and hsa-miR-6875-5p; hsa-miR-18b-3p, hsa-miR-1202, miR-3135b and hsa-miR-6769b-5p; hsa-miR-18b-3p, hsa-miR-1202, miR-3135b and hsa-miR-6875-5p; hsa-miR-18b-3p, hsa-miR-1202, hsa-miR-6769b-5p and hsa-miR-6875-5p; hsa-miR-18b-3p, miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202; hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, hsa-miR-3135b and hsa-miR-6769b-5p; hsa-miR-18b-3p, hsa-miR-494-3p, hsa-miR-1202, hsa-miR-3135b and hsa-miR-6875-5p; hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p.

In some embodiments, the biological sample of liquid biopsy is selected from the group consisting of saliva, blood, plasma, serum, preferably saliva.

In some embodiments, the antitumor therapy is selected from the group consisting of chemotherapy, radiotherapy and/or therapy with molecularly targeted drugs. In some embodiments, the chemotherapy or therapy with molecularly targeted drugs is selected from the group consisting of a PI3Kα inhibitor such as, for example, alpelisib, an alkylating agent such as, for example, cisplatin, oxaliplatin or ifosfamide, an antimetabolite such as, for example, 5-fluorouracile, capecitabin or azacytidine, an antimitotic such as, for example, paclitaxel, docetaxel or vinorelbine, an antitumour antibiotic such as, for example, doxorubicin, a topoisomerase I inhibitor such as, for example, irinotecan, an EGFR inhibitor such as, for example, cetuximab, an EGFR-TK inhibitor such as, for example, erlotinib, an mTOR inhibitor such as, for example, everolirnus.

In some embodiments, the method is carried out by Real-Time POR, Microarray or RNA Next Generation Sequencing. In some embodiments, the antitumor therapy is against a tumor selected from the group consisting of solid tumours, such as, for example, head and neck cancer, or non-solid tumours, such as lymphoma or leukaemia. In some embodiments, the mucositis is a mucositis of the oropharyngeal cavity, such as, oral mucositis.

In some embodiments, a kit for in vitro diagnosis of mucositis in a cancer patient undergoing antitumor therapy includes one or more primer pairs and/or one or more probes complementary to more than one microRNAs, where the microRNA is selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-hsa-miR-18b-3p and hsa-miR-6875-5p; hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b; hsa-miR-18b-3p, hsa-miR-1202 and hsa-miR-6769b-5p; hsa-miR-18b-3p, hsa-miR-1202 and hsa-miR-6875-5p; hsa-miR-18b-3p, hsa-miR-3135b and hsa-miR-6769b-hsa-miR-18b-3p, hsa-miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; miR-494-3p, hsa-miR-1202 and hsa-miR-6769b-miR-494-3p, hsa-miR-1202 and hsa-miR-6875-5p; miR-494-3p, hsa-miR-3135b and hsa-miR-6769b-5p; miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, miR-3135b and hsa-miR-6769b-5p; hsa-miR-18b-3p, miR-494-3p, miR-3135b and hsa-miR-6875-5p; hsa-miR-18b-3p, miR-494-3p, hsa-miR-6769b-5p and hsa-miR-6875-5p; hsa-miR-18b-3p, hsa-miR-1202, miR-3135b and hsa-miR-6769b-5p; hsa-miR-18b-3p, hsa-miR-1202, miR-3135b and hsa-miR-6875-5p; hsa-miR-18b-3p, hsa-miR-1202, hsa-miR-6769b-5p and hsa-miR-6875-5p; hsa-miR-18b-3p, miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202, hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, hsa-miR-3135b and hsa-miR-6769b-5p; hsa-miR-18b-3p, hsa-miR-494-3p, hsa-miR-1202, hsa-miR-3135b and hsa-miR-6875-5p; hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p, where the primers and/or probes include a detectable label and reagents for measuring the amount of said detectable label.

In some embodiments, a method for treatment of a tumor in a cancer patient includes obtaining a measurement, in at least one biological sample of liquid biopsy from the cancer patient, the expression of an miRNA selected from the group consisting of hsa-miR-18b-3p, hsa-miR-494-3p, hsa-miR-1202, hsa-miR-3135b, hsa-miR-6769b-5p and hsa-miR-6875-5p; subjecting the cancer patient to antitumor therapy selected from the group consisting of chemotherapy, radiotherapy and therapy with molecularly targeted drugs; obtaining a measurement, in at least one biological sample of liquid biopsy from the cancer patient after the antitumor therapy, the expression of the miRNA; and determining whether a change has occurred in the expression of said miRNAs in at least one biological sample taken after the antitumor therapy compared to the expression of said miRNAs in at least one biological sample taken before the antitumor therapy. In some embodiments, the antitumor therapy is selected from the group consisting of administering to the cancer patient a chemotherapy or drug selected from the group consisting of PI3Kα inhibitor, an alkylating agent, an antimetabolite, an antimitotic, an antitumour antibiotic, a topoisomerase I inhibitor, an EGFR inhibitor, an EGFR-TK inhibitor, and an mTOR inhibitor. In another embodiments, the antitumor therapy is selected from the group consisting of administering a chemotherapy or drug selected from the group consisting of alpelisib, cisplatin, oxaliplatin, ifosfamide, 5-fluorouracile, capecitabin, azacytidine, paclitaxel, docetaxel, vinorelbine, doxorubicin, irinotecan, cetuximab, erlotinib, and everolimus.

### DETAILED DESCRIPTION

At present, there is no evidence regarding the use of microRNAs as markers of oral mucositis. The recently published evidence showing the role of miRNA 200c in the molecular mechanism of the development of mucositis is not linkable to a diagnosis of mucositis, [13].

According to the present invention, it has now been found that six microRNAs, namely hsa-miR-18b-3p, hsa-miR-494-3p, hsa-miR-1202, hsa-miR-3135b, hsa-miR-6769b-5p and/or hsa-miR-6875-5p, are specifically correlated to the development of mucositis following antitumour treatments, such as for example chemo- or radiotherapy treatments. In particular, it has been found that the aforesaid miRNAs are also associated with the initial stage of pathogenesis, i.e. the initiation of tissue damage, making these miRNAs suitable for promptly diagnosing mucositis at a very early stage.

In greater detail, as shown by the experimental data shown further below, according to the present invention it has been observed that the six above-mentioned miRNAs are commonly deregulated in primary lines of gingival keratinocytes and epidermal keratinocytes (HEKa) originating from the oral mucosa of healthy donors treated with various antineoplastic agents, compared to untreated control samples. In particular, it was observed that the hsa-miR-18b-3p miRNA was not detectable in the control samples, whereas it was always overregulated in the treated samples, irrespective of the type of antineoplastic treatment performed. The levels of circulating hsa-miR-18-3p were then evaluated in the serum of patients affected by advanced-stage head and neck cancer (stages Ill and IV) and a significant deregulation of the hsa-miR-18b-3p miRNA was observed, both before and after the chemo- and radiotherapy treatment. These results point to the efficacy of the biomarker in the timely diagnosis of mucositis.

Although the experimentation was conducted in patients affected by head and neck cancer, the results make it plausible that the same biomarker can be adapted for the diagnosis of mucositis also in cancer patients affected by other types of solid tumour, such as for example colon and rectum adenocarcinoma, breast carcinoma, lung adenocarcinoma and lung squamous cell carcinoma and gastric adenocarcinoma. The analysis shown in FIG. 7 shows, in fact, that the expression of miR-18b-3p is not only very low in the tumour tissues analysed, but it also does not show a significant difference between healthy tissue and tumour tissue. An increase in the levels of circulating miR-18b-3p thus seems not to depend on the type of tumour, but is rather indicative of the damage to the oral mucosa induced by the antineoplastic therapy.

The method proposed according to the present invention advantageously enables an early identification of the process of pathogenesis of mucositis, such as to permit a more rapid management of the patient based on his/her clinical situation/history. Furthermore, the method according to the present invention can be advantageously used to detect the tendency of the patient to develop mucositis.

The method according to the present invention advantageously enables the detection of changes in the levels of expression of the aforesaid six circulating microRNAs in the initial phase of the process of pathogenesis. The data reported further below show that the microRNAs are differently released in the culture medium of primary lines of gingival epithelium in the first hours following treatment. Furthermore, the method according to the invention is not treatment-specific. In fact, a deregulation of the six microRNAs was revealed in all of the treatments tested.

Furthermore, the data shown further below reveal a common deregulation of the aforesaid microRNAs irrespective of the type of damage induced; therefore, the method according to the invention can be advantageously used both in subjects in whom the damage is caused by molecularly targeted drugs and in subjects in whom the damage is caused by standard antineoplastic agents.

The method according to the present invention offers a considerable advantage in cancer patient management. As noted above, said method would in fact enable a timely identification of the process of pathogenesis through monitoring of the levels of expression of at least one of the 6 microRNAs during the treatment (follow-up). This would ensure a rapid management of the pathology, such as to attenuate the symptoms thereof, thus reducing the cases of hospitahsation and the consequent increase in costs for the national health system.

The method according to the present invention is simple and non-invasive and requires an exiguous amount of a biological sample, in particular saliva and/or serum. Furthermore, the technology used to measure the level of microRNAs is reliable, highly sensitive and specific. In addition, the prognostic power of the method according to the invention would not be reduced by the non-expression of one or more of the six tested microRNAs, but would rather remain unchanged when one or more of the remaining ones are considered.

At present, there is little bibliographic evidence regarding the aforesaid 6 microRNAs and no correlations have been found between the levels of expression of the latter and the onset/development of neoplasms. This suggests that the deregulation thereof may be a specific pathogenetic characteristic of mucositis, supporting their potentiality as exclusive biomarkers for this disease.

It is therefore a specific object of the present invention a method for in vitro diagnosis of mucositis in a cancer patient undergoing antitumour therapy, said method comprising or consisting of measuring, in at least one biological sample of liquid biopsy taken before the antitumour therapy and in at least one biological sample of liquid biopsy taken after the antitumour therapy, the expression of one, more than one or all of the miRNAs selected from among hsa-miR-18b-3p (Accession Number: M10001518), hsa-miR-494-3p (Accession Number: M10003134), hsa-miR-1202 (Accession Number: M10006334), hsa-miR-3135b (Accession Number: M10016809), hsa-miR-6769b-5p (Accession Number: M10022706) and/or hsa-miR-6875-(Accession Number: M10022722), wherein a change in the expression of said miRNAs in said at least one biological sample of liquid biopsy taken after the antitumour therapy compared to the expression of said miRNAs in said at least one biological sample of liquid biopsy taken before the antitumour therapy indicates mucositis.

Therefore, according to the method of the present invention, the expression of said one, more than one or all of the microRNAs can be measured by using one or more primer pairs and/or one or more probes suitable for the purpose. It is well known that primers and probes are oligonucleotide sequences complementary to the sequences of the microRNAs to be detected.

According to the present invention, the mucositis can be a mucositis of the oropharyngeal cavity, such as, for example, oral mucositis.

Furthermore, according to the present invention, the mucositis can be in the pathogenetic stage of initiation of tissue damage. Therefore, the method according to the present invention is capable of diagnosing mucositis at an early stage, as well as in the subsequent stages of pathogenesis of the disease.

According to one embodiment of the method according to the present invention, said one, more than one or all of the miRNAs comprise or consist of hsa-miR-18b-3p. The method according to the present invention, according to one embodiment, preferably uses at least hsa-miR-18b-3p, since it is not present before the antitumour treatment and is always overregulated following any antitumour treatment. Therefore, according to the present invention, the step of measuring the expression of one, more than one or all said miRNAs can be carried out only in said at least one biological sample taken after the antitumour therapy (i.e. not in a biological sample taken before the antitumour therapy), when said one, more than one or all of the miRNAs consist of hsa-miR-18b-3p.

According to the present invention, said more than one or all of the miRNAs can be selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-5p; hsa-miR-18b-3p and hsa-miR-6875-5p; hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b; hsa-miR-18b-3p, hsa-miR-1202 and hsa-miR-6769b-5p; hsa-miR-18b-3p, hsa-miR-1202 and hsa-miR-6875-5p; hsa-miR-18b-3p, hsa-miR-3135b and hsa-miR-6769b-5p; hsa-miR-18b-3p, hsa-miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; miR-494-3p, hsa-miR-1202 and hsa-miR-6769b-5p; miR-494-3p, hsa-miR-1202 and hsa-miR-6875-5p; miR-494-3p, hsa-miR-3135b and hsa-miR-6769b-5p; miR-494-3p, hsa-miR-3135b and hsa-miR-6875-5p; hsa-miR-494-3p; hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, miR-3135b and hsa-miR-6769b-5p; hsa-miR-18b-3p, miR-494-3p, miR-3135b and hsa-miR-6875-5p; hsa-miR-18b-3p, miR-494-3p, hsa-miR-6769b-5p and hsa-miR-6875-5p; hsa-miR-18b-3p, hsa-miR-1202, miR-3135b and hsa-miR-6769b-5p; hsa-miR-18b-3p, hsa-miR-1202, miR-3135b and hsa-miR-6875-5p; hsa-miR-18b-3p, hsa-miR-1202, hsa-miR-6769b-5p and hsa-miR-6875-5p; hsa-miR-18b-3p, miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202, hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, hsa-miR-3135b and hsa-miR-6769b-5p; hsa-miR-18b-3p, hsa-miR-494-3p, hsa-miR-1202, hsa-miR-3135b and hsa-miR-6875-5p; hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p.

According to the present invention, said biological sample of liquid biopsy can be a sample of saliva, blood, plasma, serum, preferably of saliva. Preferably, the measurement of the expression of said one, more than one or all of the above-mentioned mRNAs is performed within the serum and/or the saliva originating from patients affected by cancer, such as, for example, head and neck cancer, who will undergo and/or have undergone chemo- and/or radiotherapy treatment. In particular, the levels of said one, more than one or all of the above-mentioned miRNAs will preferably be measured, for each patient, before the antitumour treatment (baseline) and after every antitumour treatment cycle, for example after about three days. The biological sample is preferably processed within an hour after being collected and subsequently treated for RNA extraction.

According to the present invention, said antitumour therapy can be selected from the group consisting of chemotherapy, radiotherapy and/or a therapy with molecularly targeted drugs, such as, for example, mTOR inhibitors. In particular, the antitumour therapy can comprise the use of a PI3Kα inhibitor such as, for example, alpelisib, an alkylating agent such as, for example, cisplatin, oxaliplatin or ifosfamide, an antimetabolite such as, for example, 5-fluorouracile, capecitabin or azacytidine, an antimitotic such as, for example, paclitaxel, docetaxel or vinorelbine, an antitumour antibiotic such as, for example, doxorubicin, a topoisomerase I inhibitor such as, for example, irinotecan, an EGFR inhibitor such as, for example, cetuximab, an EGFR-TK inhibitor such as, for example, erlotinib, or an mTOR inhibitor such as, for example, everolirnus.

Furthermore, according to the present invention, said antitumour therapy can be against a tumour selected from the group consisting of a solid tumour, such as, for example, head and neck cancer, or a non-solid tumour, such as lymphoma or leukaemia.

The method according to the present invention can be carried out by Real Time PCR, Microarray o RNA Next Generation Sequencing.

The detection of a change in the expression of the miRNAs according to the present invention would guarantee a prompt intervention by the clinician, who, through a close observation of the patient, will be able to suggest specific treatments making reference to the guidelines for intervention published by the Multinational Association of Supportive Care in Cancer and International Society for Oral Oncology (MASCC/ISOO) based on evidence regarding the prevention and treatment of mucositis caused by cancer therapies [14], and/or, on the basis of the degree of mucositis, to opt to increase the interval of time between one treatment cycle and the next, and/or to possibly change the dosage of the antineoplastic treatment. Therefore, the present invention further relates to a method for the antitumour treatment of a subject undergoing antitumour therapy, said method comprising

measuring, in at least one biological sample of liquid biopsy taken before the antitumour therapy and in at least one biological sample of liquid biopsy taken after the antitumour therapy, the expression of one, more than one or all of the miRNAs selected from among hsa-miR-18b-3p (Accession Number: M10001518), hsa-miR-494-3p (Accession Number: M10003134), hsa-miR-1202 (Accession Number: M10006334), hsa-miR-3135b (Accession Number: M10016809), hsa-miR-6769b-5p (Accession Number: MI0022706) and/or hsa-miR-6875-5p (Accession Number: M10022722) and,

when a change is observed in the expression of said miRNAs in said at least one biological sample of liquid biopsy taken after the antitumour therapy compared to the expression of said miRNAs in said at least one biological sample of liquid biopsy taken before the antitumour therapy, following one or more of the following options: administering specific treatments for the prevention and treatment of mucositis, such as, for example, the ones indicated in the guidelines provided by the Multinational Association of Supportive Care in Cancer and International Society for Oral Oncology (MASCC/ISOO), increasing the interval of time between one treatment cycle and the next, and/or varying the dosage of the antineoplastic treatment.

A further object of the present invention is the use of one, more than one or all of the microRNAs selected from among hsa-miR-18b-3p, hsa-miR-494-3p, hsa-miR-1202, hsa-miR-3135b, hsa-miR-6769b-5p and/or hsa-miR-6875-5p as biomarkers for the in vitro diagnosis of mucositis caused by antitumour therapy. As said above, the mucositis can be a mucositis of the oropharyngeal cavity, such as oral mucositis. Furthermore, the mucositis can be in the pathogenetic stage of initiation of tissue damage.

According to one embodiment of the present invention, said one, more than one or all of the miRNAs comprise or consist of hsa-miR-18b-3p.

Said more than one or all of the miRNAs can be selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-5p; hsa-miR-18b-3p and hsa-miR-6875-5p; hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and miR-3135b; hsa-miR-18b-3p, hsa-miR-1202 and hsa-miR-6769b-5p; hsa-miR-18b-3p; hsa-miR-1202 and hsa-miR-6875-5p; hsa-miR-18b-3p, miR-3135b and hsa-miR-6769b-5p; hsa-miR-18b-3p, miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; hsa-miR-494-3p, hsa-miR-1202 and hsa-miR-6769b-5p; hsa-miR-494-3p, hsa-miR-1202 and hsa-miR-6875-5p; hsa-miR-494-3p, hsa-miR-3135b and hsa-miR-6769b-5p; hsa-miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, hsa-miR-3135b and hsa-miR-6769b-5p; hsa-miR-18b-3p, hsa-miR-494-3p, hsa-miR-3135b and hsa-miR-6875-5p; hsa-miR-18b-3p, hsa-miR-494-3p, hsa-miR-6769b-5p and hsa-miR-6875-5p; hsa-miR-18b-3p, hsa-miR-1202, hsa-miR-3135b and hsa-miR-6769b-5p; hsa-miR-18b-3p, hsa-miR-1202, hsa-miR-3135b and hsa-miR-6875-5p; hsa-miR-18b-3p; hsa-miR-1202, hsa-miR-6769b-5p and hsa-miR-6875-5p; hsa-miR-18b-3p, hsa-miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202, hsa-miR-6769b-5p and hsa-miR-6875-5p; hsa-miR-494-3p, hsa-miR-3135b; hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, miR-3135b and hsa-miR-6769b-5p; hsa-miR-18b-3p, hsa-miR-494-3p, hsa-miR-1202, miR-3135b and hsa-miR-6875-5p; hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p.

According to the present invention, the antitumour therapy can be selected from the group consisting of chemotherapy, radiotherapy and/or therapy with molecularly targeted drugs, such as, for example, mTOR inhibitors.

In particular, the antitumour therapy can comprise the use of a PI3Kα inhibitor such as, for example, alpelisib, an alkylating agent such as, for example, cisplatin, oxaliplatin or ifosfamide, an antimetabolite such as, for example, 5-fluorouracile, capecitabin or azacytidine, an antimitotic such as, for example, paclitaxel, docetaxel or vinorelbine, an antitumour antibiotic such as, for example, doxorubicin, a topoisomerase I inhibitor such as, for example, irinotecan, an EGFR inhibitor such as, for example, cetuximab, an EGFR-TK inhibitor such as, for example, erlotinib, an mTOR inhibitor such as, for example, everolimus.

Furthermore, according to the present invention, said antitumour therapy can be against a tumour selected from the group consisting of solid tumours, such as, for example, head and neck cancer, or non-solid tumours, such as lymphoma or leukaemia.

As said above, according to the present invention the mucositis can be a mucositis of the oropharyngeal cavity, such as, for example, oral mucositis.

The present invention also relates to a kit for in vitro diagnosis of mucositis in a cancer patient undergoing antitumour therapy, said kit comprising or consisting of one or more primer pairs and/or one or more probes for the detection of one, more than one or all of the microRNAs selected from among hsa-miR-18b-3p, hsa-miR-494-3p, hsa-miR-1202, hsa-miR-3135b, hsa-miR-6769b-5p and/or hsa-miR-6875-5p. According to one embodiment, the kit of the present invention does not comprise primers and/or probes for the detection of miRNAs other than hsa-miR-18b-3p, hsa-miR-494-3p, hsa-miR-1202, hsa-miR-3135b, hsa-miR-6769b-5p and hsa-miR-6875-5p.

According to the present invention, said more than one or all of the miRNAs can be selected from the group consisting of hsa-miR-18b-3p and miR-494-3p; hsa-miR-18b-3p and hsa-miR-1202; hsa-miR-18b-3p and hsa-miR-3135b; hsa-miR-18b-3p and hsa-miR-6769b-5p; hsa-miR-18b-3p and hsa-miR-6875-5p; miR-494-3p and hsa-miR-1202; miR-494-3p and hsa-miR-3135b; miR-494-3p and hsa-miR-6769b-5p; miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b; hsa-miR-18b-3p, hsa-miR-1202 and hsa-miR-6769b-5p; hsa-miR-18b-3p, hsa-miR-1202 and hsa-miR-6875-5p; hsa-miR-18b-3p, hsa-miR-3135b and hsa-miR-6769b-5p; hsa-miR-18b-3p, hsa-miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; hsa-miR-494-3p, hsa-miR-1202 and hsa-miR-6769b-5p; hsa-miR-494-3p, hsa-miR-1202 and hsa-miR-6875-5p; hsa-miR-494-3p, hsa-miR-3135b and hsa-miR-6769b-hsa-miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, hsa-miR-3135b and hsa-miR-6769b-5p; hsa-miR-18b-3p, hsa-miR-494-3p, hsa-miR-3135b and hsa-miR-6875-5p; hsa-miR-18b-3p, hsa-miR-494-3p, hsa-miR-6769b-5p and hsa-miR-6875-5p; hsa-miR-18b-3p, hsa-miR-1202, hsa-miR-3135b and hsa-miR-6769b-5p; hsa-miR-18b-3p, hsa-miR-1202, hsa-miR-3135b and hsa-miR-6875-5p; hsa-miR-18b-3p, hsa-miR-1202, hsa-miR-6769b-5p and hsa-miR-6875-5p; hsa-miR-18b-3p, hsa-miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202, hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, miR-494-3p, hsa-miR-1202, hsa-miR-3135b and hsa-miR-6769b-5p; hsa-miR-18b-3p, hsa-miR-494-3p, hsa-miR-1202, hsa-miR-3135b and hsa-miR-6875-5p; hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p. According to one embodiment, the kit of the present invention comprises primers and probes solely for the detection of the miRNAs mentioned above and not of other miRNAs.

### BRIEF DESCRIPTION OF THE DRAWINGS

The present invention will now be described, by an illustrative but not limitative way, according to a preferred embodiment thereof, with particular reference to the examples and the figures in the appended drawings, wherein;

FIG. 1 shows the treatment with Group 1, drugs with an alkylating action. Viability curves drawn at 72 hours on the primary line of gingival keratinocytes and epidermal keratinocytes (HEKas) treated with increasing doses of Cisplatin (A), Oxaliplatin (C) and Ifosfamide (D) (ATPlite Assay, Perkin Elmer). (B) impedance-based assay conducted on the HEKa line treated with different doses of Cisplatin for 1200 minutes (Label-Free Technology, Perkin Elmer);

FIG. 2 shows the treatment with Group 2, anti-metabolite drugs and topoisomerase inhibitors. Viability curves drawn at 72 hours on the primary line of gingival keratinocytes and HEKas treated with increasing doses of 5-fluoracile (A), Irinotecan (C) (FIG. 2a), Azacytidine (E) and Capecitabin (G) (FIG. 2b) (ATPlite Assay, Perkin Elmer). (B, D, F) impedance-based assay conducted on the HEKa line treated with different doses of 5-Fluorouracile (B), Irinotecan (D) (FIG. 2a) and Azacytidine (F) (FIG. 2b) for 1200 minutes (Label-Free Technology, Perkin Elmer);

FIG. 3 shows the treatment with Group 3, drugs with molecular action. Viability curves drawn at 72 hours on the primary line of gingival keratinocytes and HEKas treated with increasing doses of Erlotinib (A), Cetuximab (C) (FIG. 3a), Everolimus (E) and Alpelisib (G) (FIG. 3b) (ATPlite Assay, Perkin Elmer). (B, D, F, H) impedance-based assay conducted on the HEKa line treated with different doses of Erlotinib (B), Cetuximab (D) (FIG. 3a) Everolimus (F) and Alpelisib (H) (FIG. 3b) for 1200 minutes (Label-Free Technology, Perkin Elmer);

FIG. 4 shows the treatment with Group 4, antibiotic and antimitotic drugs. Viability curves drawn at 72 hours on the primary line of gingival keratinocytes and HEKas treated with increasing doses of Docetaxel (A), Vinorelbine (C)(FIG. 4a), Doxorubicin (E) and Paclitaxel (G) (FIG. 4b) (ATPlite Assay, Perkin Elmer), (B, D, F) impedance-based assay conducted on the HEKa line treated with different doses of Docetaxel (B), Vinorelbine (D) (FIG. 4a) and Paclitaxel (F) (FIG. 4b) for 1200 minutes (Label-Free Technology, Perkin Elmer);

FIG. 5 shows (A) principal component analysis (PCA) of the differently treated samples (FIG. 5a) (B) unsupervised hierarchical clustering with Euclidean metrics correlating the expression of the circulating microRNAs present in the culture media of the primary line of gingival keratinocytes with the different treatments (FIG. 5a). (C) Venn diagram relating to 99 microRNAs found to be differently expressed in at least one of the four groups versus the control. 6 of these are common to all of the groups analysed (FIG. 5a). (D) Box plot of the levels of expression, shown on a logarithmic scale, of the 6 microRNAs differently modulated versus the control in the four groups analysed (FIG. 5b);

FIG. 6 shows (A) cycles of detection of the expression levels of the hsa-miR-18b-3p miRNA (cycle threshold) (FIG. 6a). (B) analysis of the changes in the expression levels of the hsa-miR-18b-3p miRNA before and after radiotherapy treatment (FIG. 6a). (C) design of pilot study relating to the identification of the circulating miRNAs correlated to the process of cachexia in patients with head and neck cancer (stages Ill and IV) undergoing combined chemoradiotherapy treatment (FIG. 6b). (D) analysis of the changes in the expression levels of the hsa-miR-18b-3p miRNA before and after radiotherapy treatment and three months after the last cycle of therapy (FIG. 6b). The differences in expression are in relation to baseline levels. Statistics, T-Test: *≤0.05.

FIG. 7 shows the expression levels originating from RNA-seg data expressed in RPM (reads per million mapped reads) of the miR-18b-3p that may be found among the cases deposited in the public database of the “The Cancer Genome Atlas” (TOGA) in healthy tissue (con) and in tumour tissue (turn) of patients affected by head and neck cancer (con: 15, turn: 134)(A), colon adenocarcinoma (con: 3, turn: 33)(B) and rectum adenocarcinoma (con: 3, turn: 14)(C), breast carcinoma (con: 24, turn: 134)(D), lung adenocarcinoma (con: 15, turn: 150)(E) and lung squamous cell carcinoma (con: 26, turn: 118)(F) and gastric adenocarcinorna (con: 17, turn: 93)(G).

### EXAMPLE 1: IN VITRO STUDY FOR THE SELECTION OF THE MIRNAS ACCORDING TO THE PRESENT INVENTION FOR THE EARLY DIAGNOSIS OF MUCOSITIS IN CANCER PATIENTS

Materials and Methods

Cell Cultures and Treatments

Use was made of two primary cells lines of keratinocytes originating from gums (Primary Gingival Keratinocytes ATCO® PCS-200-014™) or epidermis (Primary Epidermal Keratinocytes; Normal, Human, Adult, HEKa, ATCC® PCS-200-011™) and purchased from ATCC, The two lines were cultured in Dermal Cell Basal Medium (ATCC® PCS-200-030™), to which certain components (table 1) contained in the Keratinocyte Growth Kit (ATCC® PCS-200-040™) were added. Table 1 shows the composition of the Keratinocyte Growth Kit (ATCC® PCS-200-040™).

TABLE 1

| | | |
|---|---|---|
| | | Final |
| Component | Volume | concentration |

| Extract P | 2.0 | mL | 0.4% | |
|---|---|---|---|---|
| rh TGF-a | 0.5 | mL | 0.5 | ng/mL |
| L-Glutamine | 15.0 | mL | 6 | mM |
| Hydrocortisone | 0.5 | mL | 100 | ng/mL |
| rh Insulin | 0.5 | mL | 5 | μg/mL |
| Epinephrine | 0.5 | mL | 1.0 | μM |
| Apo-Transferrin | 0.5 | mL | 5 | μg/mL |

The following synthetic antineoplastic drugs were used to treat the cells: alpelisib (Selleckchem—S2814), cisplatin (Selleckchern—S1166), 5-fluorouracile (Selleckchem—S1209), paclitaxel (Selleckchem—S1150), docetaxel (Selleckchem—S1148), doxorubicin (Selleckchem—S1208), oxaliplatin (Selleckchem—S1224), capecitabin (Selleckchem—S1156), irinotecan (Selleckchem—S2217), vinorelbine (Selleckchem—S4269), Ifosfamide (Selleckchem—S1302), azacytidine (Selleckchem—S1782), cetuximab (Selleckchem—A2000), erlotinib (Selleckchem—S7786), everolimus (Selleckchem—S1120). The drugs were reconstituted in 100% DMSO according to the manufacturer's directions, to a final concentration of 10 mM.

Cell Viability Assays

In order to assess cell viability following treatment, 5000 cells/100 μl per spot were plated in a 96-well plate. The next day the following treatments were added: alpelisib (0.000005; 0,00005; 0.0005; 0.005; 0.05; 0.5 and 5 μM), cisplatin (32, 16, 8, 4, 2, 1 and 0.5 μM), 5-fluorouracils (0.03; 0.06; 0.12; 0.25; 0.5; 1 and 10 μM), paclitaxel (0.000001; 0.00001; 0.0001; 0.001; 0.01; 0.1 and 1 μM), docetaxel (0.000001; 0.00001; 0.0001; 0.001; 0.01; 0.1 and 1 μM), doxorubicin (0.0312; 0.0625; 0.125; 0.25; 0.5; 1 and 2 μM), oxaliplatin (0.78; 2; 6.25; 12.5; 16 and 32 μM), capecitabin (0.3; 0.6; 1.25; 2.5; 5; 10 and 100 μM), irinotecan (1.5; 3; 6.25; 12.5; 25; 50 and 100 μM), vinorelbine (0.000001; 0.00001; 0.0001; 0.001; 0.01; 0.1 and 1 μM), ifosfamide (0.119; 0.23; 0.47; 0.95; 1.9 and 3.83 μM), azacytidine (0.03; 0.06; 0.12; 0.25; 0.5; 1 and 10 μM), cetuximab (0.000005; 0.00005; 0.0005; 0.005; 0.05; 0.5 and 5 μM), erlotinib (0.15; 0.3; 0.6; 1.25; 2.5; 5; 12.5; 25 and 50 μM), and everolimus (0.000005; 0,00005; 0.0005; 0.005; 0.05; 0.5 and 5 μM). After 72 hours of treatment, viability was measured using the ATPlite assay (Perkin Elmer, Massachusetts, USA), following the manufacturer's directions. Each plate was then read in the luminescence mode using the EnSpire NAultimode Plate Reader (Perkin Elmer). Calcusyn software was used to calculate the CC50 value.

Slide Hybridization “microRNA Microarray”

The concentration and quality of the extracted RNA was assessed by using a Nanodrop™ 1000 spectrophotometer (Nanodrop Technologies). 100 rig of total RNA per sample were labelled and hybridised on “SurePrint G3 miRNA microarray plates, human version 21, 8×60K”, following the supplier's instructions. The signal was detected by means of an Agilent scanner and the value of the signal was extracted by means of the “feature extraction” software.

Statistical Analysis

The bioinformatic analysis was carried out using Matlab software (The MathWorks Inc.). The transformation of the signal into a “Z score” was used to express the intensity value of the spot from which the basic signal was subtracted. The characteristics were selected on the basis of the calculated Z ratios, taking the difference between the means of the observed microRNA Z scores and dividing by the standard deviation of all the differences for that particular comparison. A Z ratio greater than 1.96 was deemed significant. Unsupervised hierarchical clustering was used to examine the samples. Student's t-test was used to assess the significance of expression of the hsa-miR-18b-3p miRNA under the different experimental conditions.

Label-Free Cell Assay

HEKa cells were plated on particular 384-well plates specifically made with high precision optical sensors. The latter are capable of measuring of the changes in light refraction as a direct consequence of the dynamic redistribution of the mass within the cell culture in response to the treatment. The pm is used as the response unit and is measured in real time by using the EnSpire multimode reader (Perkin Elmer).

Biological Samples

The plasma samples used came from a collection of samples related to a non-profit clinical study entitled: “Identificazione di nuovi biomarcatori circolanti per la cachessia in pazienti affetti da tumori della testa e del collo trattati con radio-chemioterapia” (Identification of New Circulating Biomarkers for Cachexia in Patients Affected by Head and Neck Cancer Treated with Radiochemotherapy), approved by the ethics committee of the IFO (Istituti Fisioterapici Ospitalieri) on Jul. 11, 2019 with resolution number 977. The enrolled patients are affected by histologically confirmed squamous cell carcinoma of the oral cavity, oropharynx, hypopharynx and larynx at stage III or IV (cT1-4N1M0 or cT4NOMO) according to the UICC. They were treated with definitive or adjuvant concomitant radiochemotherapy (cisplatin); they are aged >18 yrs and signed the informed consent form. The biological sample, collected before, after and three months after treatment, was processed and stored in the biobank of the institute (IFO).

Results

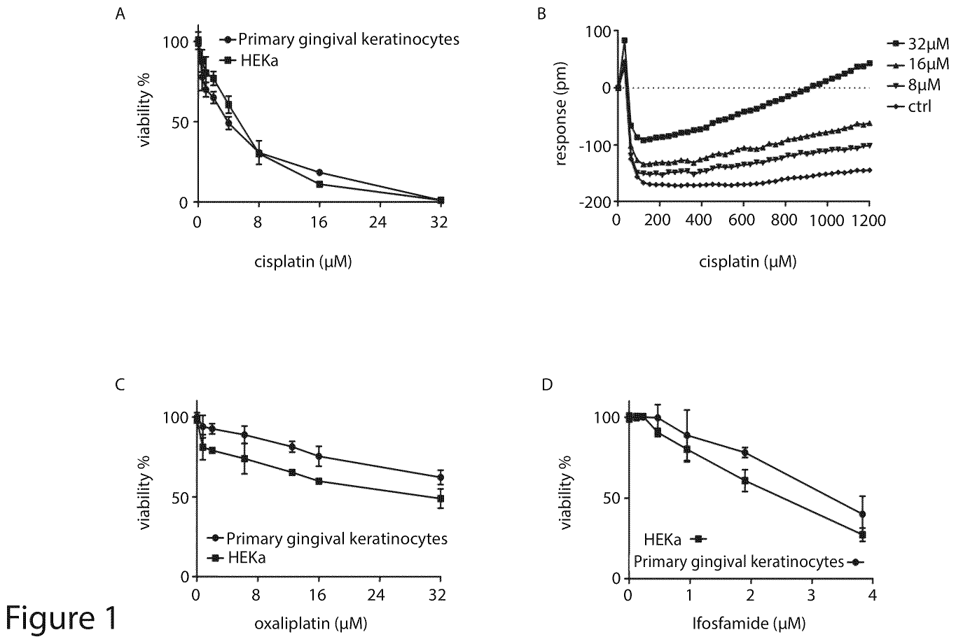

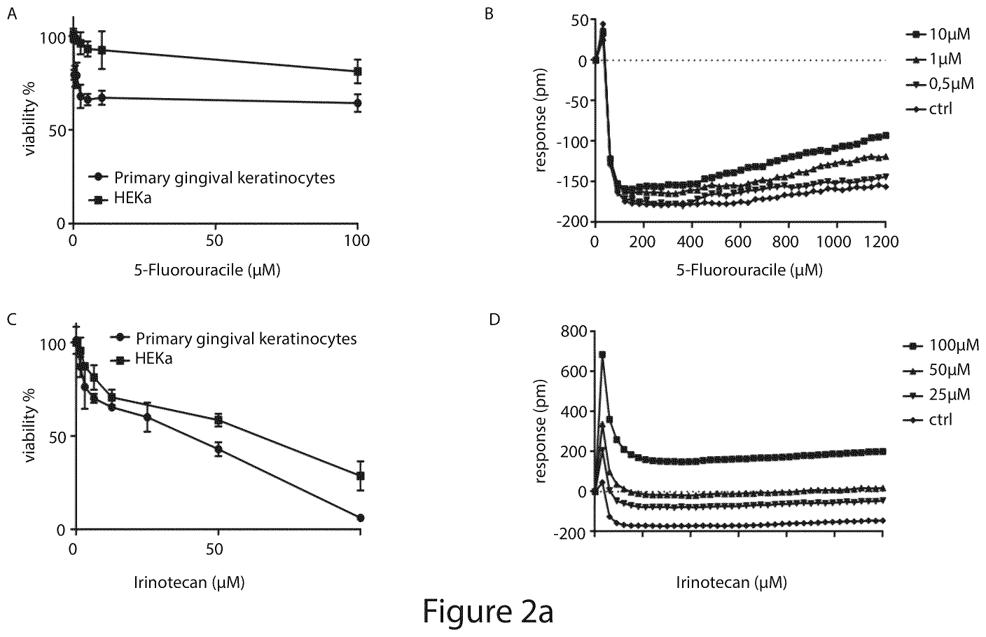

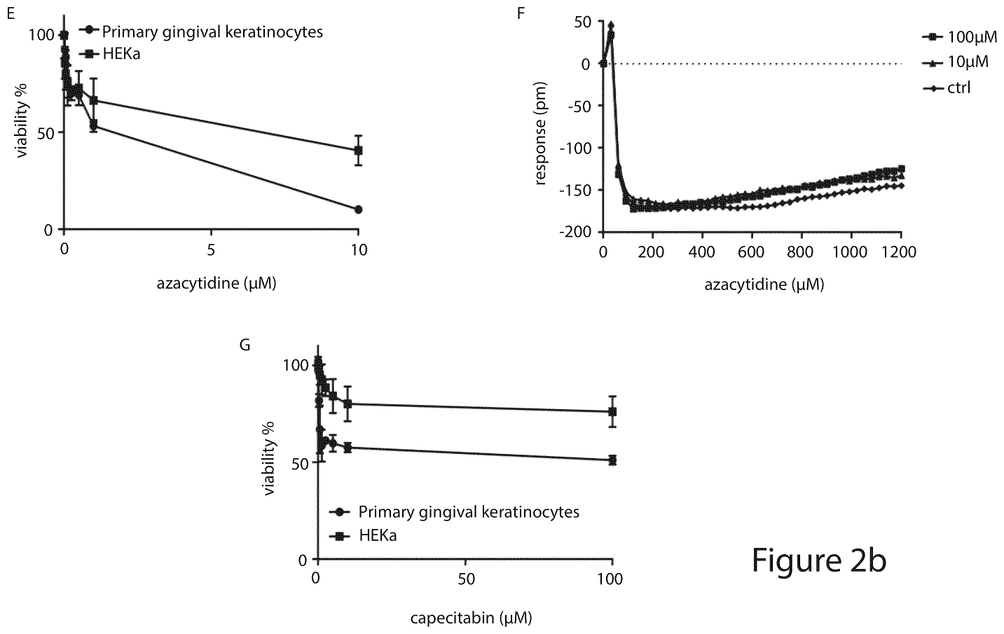

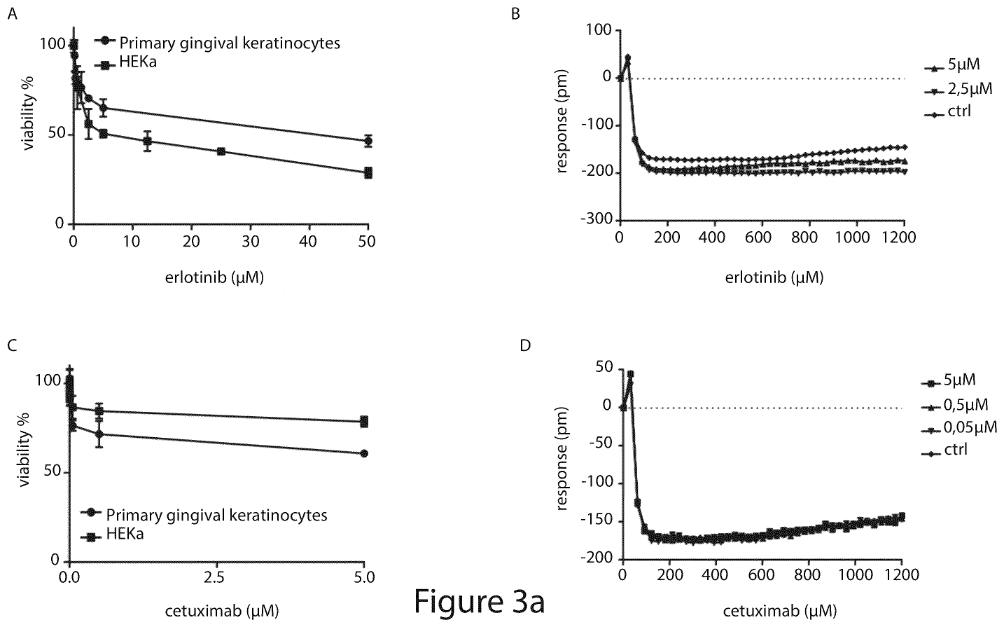

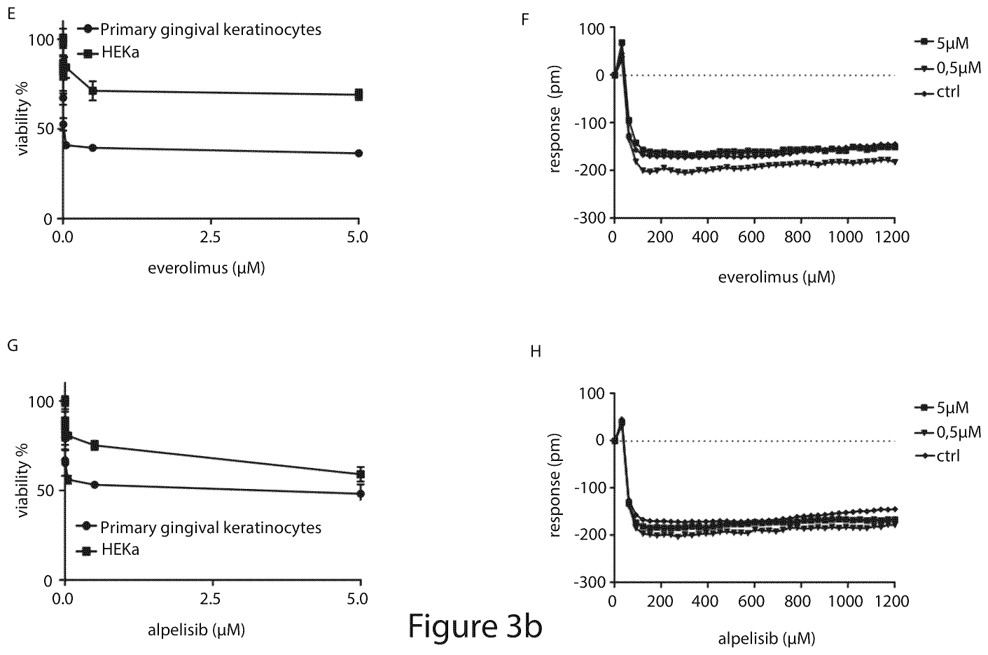

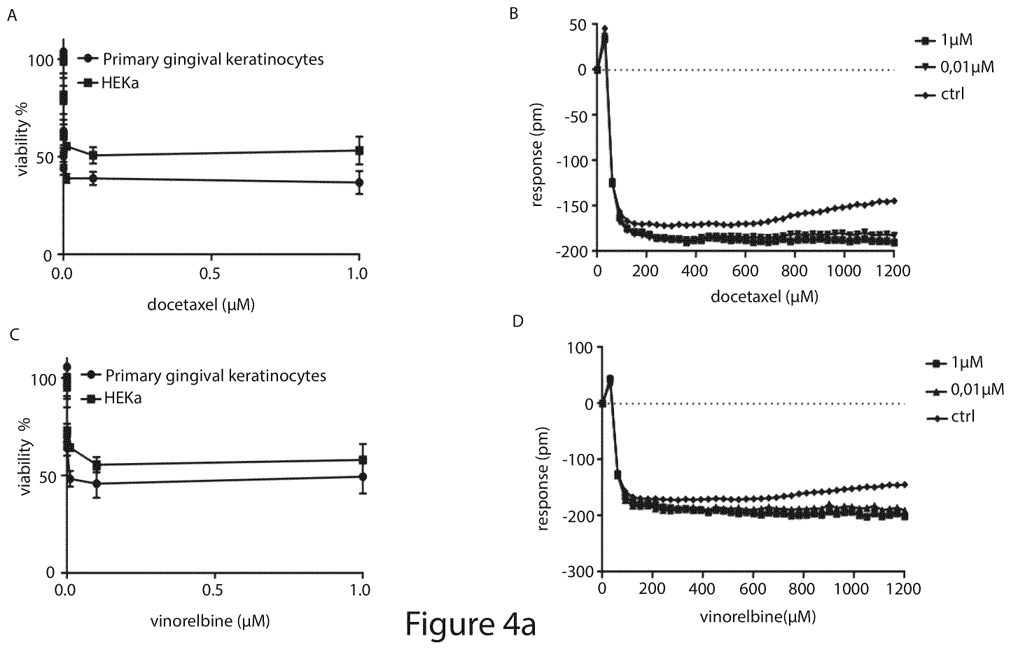

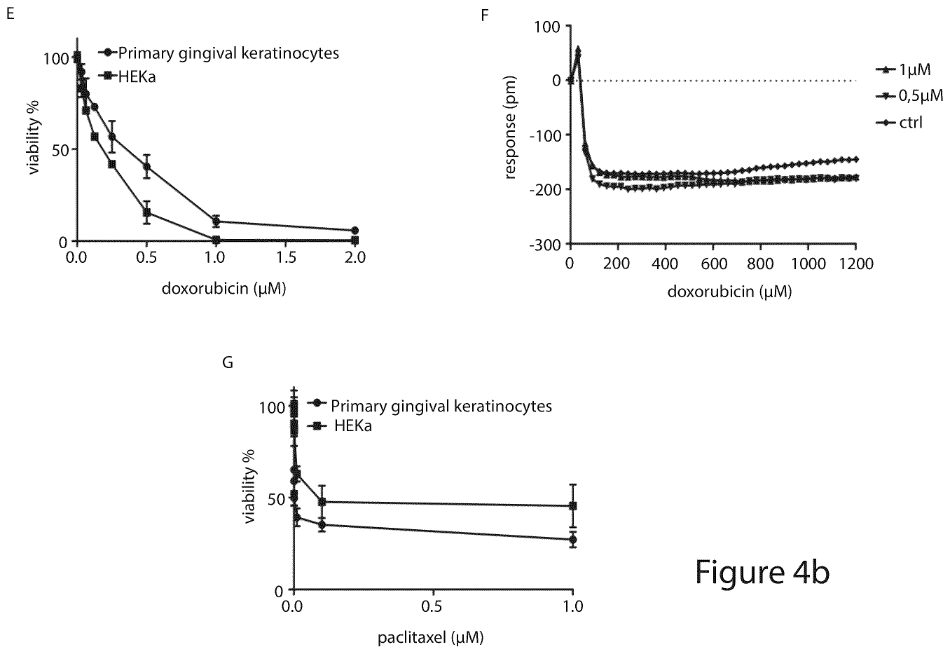

In order to mimic the first phase of the process; a primary line of gingival keratinocytes and one of epidermal keratinocytes (HEKas) were treated in vitro with different antineoplastic agents presently used in the management of HNSCC patients or tested in clinical studies (clinicaltrials.gov) (FIG. 1-4). Viability assays were conducted for each cell line at 72 hours (ATPlite assay) and over a short period (0-1200 minutes, label-free assay) using different doses of the individual drug so as to identify the sub-apoptotic dosage window which does not induce the death of more than 20% of the cell population. In this regard, the cytotoxic concentration that causes the death of 50% of the treated cells (CC₅₀) was calculated for every drug using CompuSyn software (FIG. 1-4). On the basis of the data obtained, ten representative drugs were selected and the following treatment dosages were identified for each: Cisplatin 0.5 μM, 5-Fluorouracile 0.5 μM, Irinotecan 6.25 μM, Azacytidine 0.1 μM, Erlotinib 2.5 μM, Everolimus 0.5 nM, Cetuximab 0.5 μM, Docetaxel 1 μM, Doxorubicin 0.12 μM and Alpelisib 5 nM. The culture media of the gingival keratinocytes treated for 72 hours were recovered and subjected to extraction of the total circulating RNA. 200 ng of this was then used to hybridise two Agilent slides with eight slots, each containing the specific probes for 2549 presently known microRNAs. Using the Agilent scanner it was then possible to measure the levels of intensity of each signal generated by the binding between the probe and microRNA. The subsequent analyses were conducted by dividing the various treatments into four groups on the basis of their mechanism of action.

Tables 2, 3, 4 and 5 reproduced below respectively show the composition of treatment Group 1, Group 2, Group 3 and Group 4 and give the CC50 value obtained by means of CompuSyn software on the basis of the viability curves drawn at 72 hours on the primary line of gingival keratinocytes and HEKa treated with increasing doses of each drug (FIG. 1-4).

TABLE 2

| | | CC50 Value | | | |
|---|---|---|---|---|---|
| | | Primary | | | |
| | | epidermal | | Primary | |
| GROUP 1 | Drug | keratinocytes | | gingival | |
| Drugs | description | (HEKa) | | keratinocytes | |
| CISPLATIN | Alkylating | 3 | μM | 3.8 | μM |
| | agent - | | | | |
| | Metal salts | | | | |
| OXALIPLATIN | Alkylating | 32.3 | μM | 46 | μM |
| | agent - | | | | |
| | Metal salts | | | | |
| IFOSFAMIDE | Alkylating | 2 | mM | 3.75 | mM |
| | agent - Mustard | | | | |
| | gas derivatives | | | | |

TABLE 3

| | | CC50 Value | | | |
|---|---|---|---|---|---|
| | | Primary | | | |
| | | epidermal | | Primary | |
| GROUP 2 | Drug | keratinocytes | | gingival | |
| Drugs | description | (HEKa) | | keratinocytes | |
| CAPECITABIN | Antimetabolites - | Not calculable | | 103 | μM |
| | pyrimidine | | | | |
| | antagonist | | | | |
| AZACYTIDINE | Antimetabolites - | 51.2 | μM | 1.8 | μM |
| | cytosine analogue | | | | |
| 5- | Antimetabolites - | Not calculable | | Not calculable | |
| FLUOROURACILE | pyrimidine | | | | |
| | analogue | | | | |
| IRINOTECAN | Topoisomerase I | 43 | μM | 28 | μM |
| | inhibitor | | | | |

TABLE 4

| | | CC50 Value | | | |
|---|---|---|---|---|---|
| | | Primary | | | |
| | | epidermal | | Primary | |
| GROUP 3 | Drug | keratinocytes | | gingival | |
| Drugs | description | (HEKa) | | keratinocytes | |
| ERLOTINIB | Molecular drug - | 7.8 | μM | 55 | μM |
| | EGFR-TK | | | | |
| | inhibitor | | | | |
| EVEROLIMUS | Molecular drug - | 22 | μM | 0.8 | nM |
| | MTOR inhibitor | | | | |
| CETUXIMAB | Molecular drug - | Not calculable | | 12 | μM |
| | EGFR inhibitor | | | | |
| ALPELISIB | Molecular drug - | 8 | μM | 2.5 | μM |
| | PI3Kα inhibitor | | | | |

TABLE 5

| | | CC50 Value | | | |
|---|---|---|---|---|---|
| | | Primary | | | |
| | | epidermal | | Primary | |
| GROUP 4 | Drug | keratinocytes | | gingival | |
| Drugs | description | (HEKa) | | keratinocytes | |
| PACLITAXEL | Plant alkaloid | 1 | μM | 0.001 | μM |
| | antimitotic - | | | | |
| | Taxanes | | | | |
| DOCETAXEL | Plant alkaloid | 1 | μM | 0.01 | nM |
| | antimitotic - | | | | |
| | Taxanes | | | | |
| VINORELBINE | Plant alkaloid | 0.26 | μM | 0.009 | μM |
| | antimitotic - Vinca | | | | |
| | alkaloid | | | | |
| DOXORUBICIN | Antitumour | 0.116 | μM | 0.34 | μM |
| | antibiotic - | | | | |
| | Anthracycline | | | | |

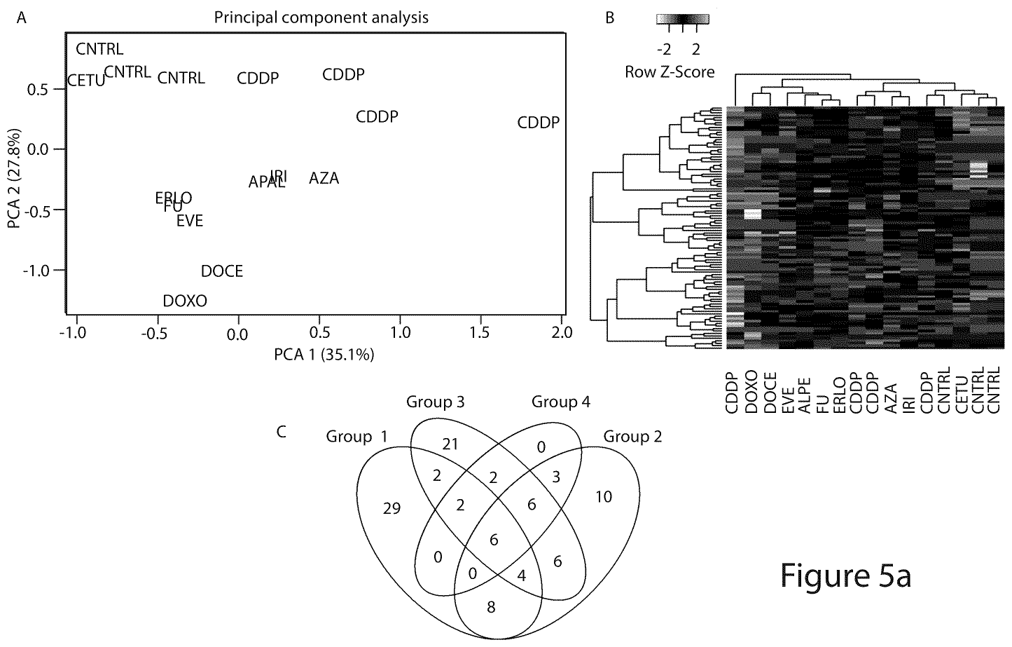

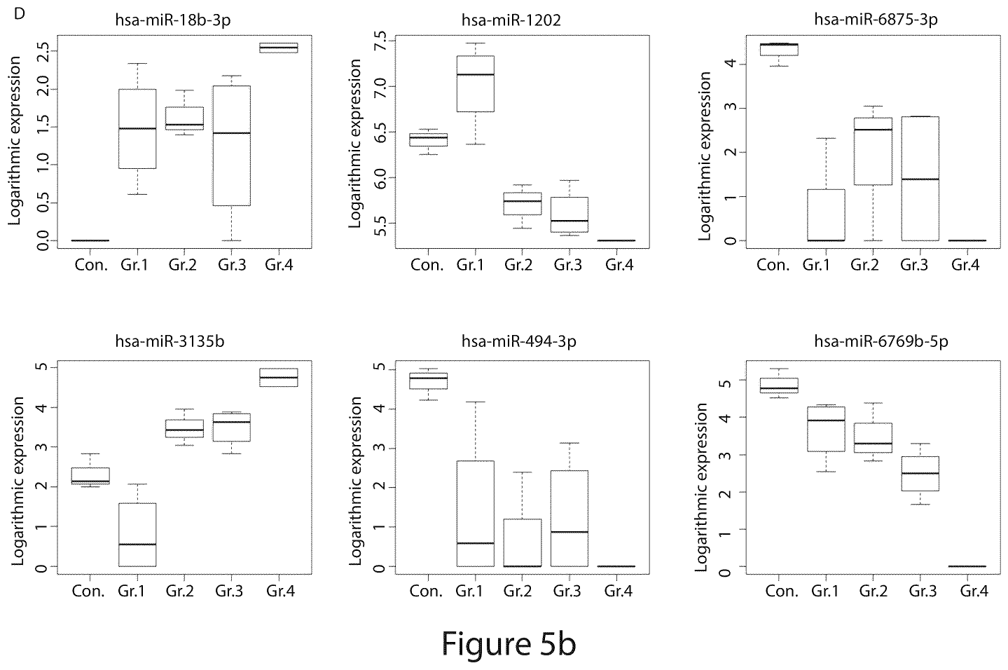

The signals were quantile-normalised and the unexpressed microRNAs excluded from subsequent analyses. The principal component analysis revealed a substantial difference between the treatment groups compared to the control samples (FIG. 5A). The result was then confirmed by unsupervised hierarchical clustering (FIG. 5B). Each treatment group was then compared with the control in order to identify the significantly deregulated microRNAs and subsequently compared with the other groups. In this manner it was possible to identify six microRNAs that were commonly deregulated across the different groups compared to the control (hsa-miR-18b-3p, hsa-miR-494-3p, hsa-miR-1202, hsa-miR-3135b, hsa-miR-6769b-5p, hsa-miR-6875-5p) (FIGS. 5C-D).

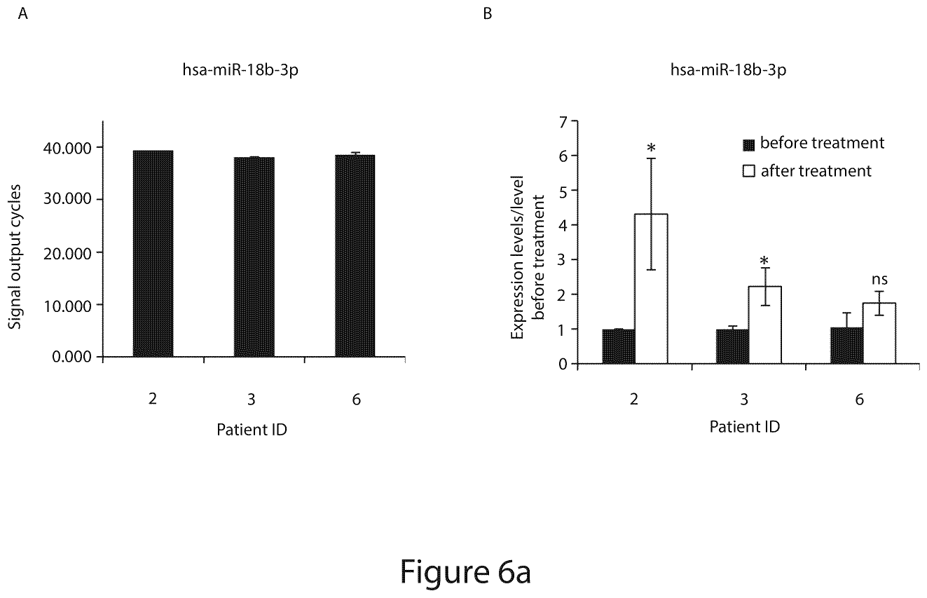

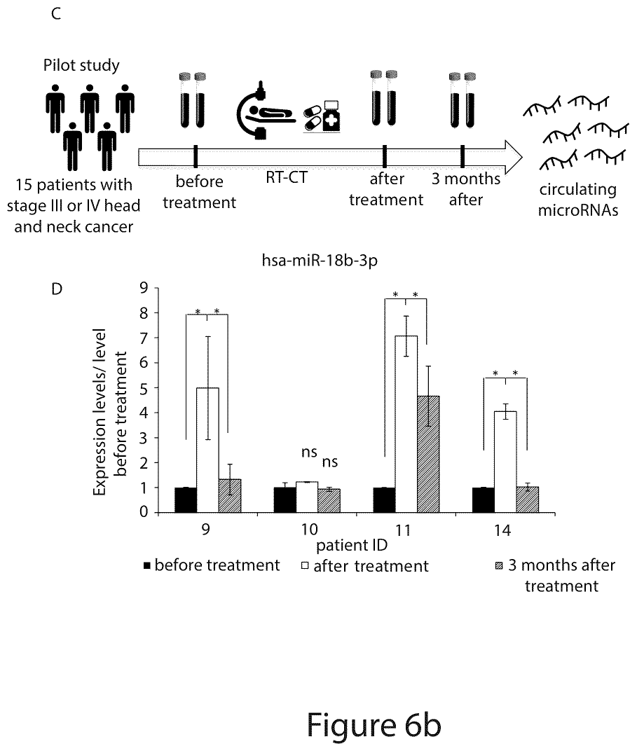

Among them, in all of the tested culture media the levels of the hsa-miR-18b-3p miRNA appeared overregulated compared to the control irrespective of the type of treatment performed. An assessment was then made of the possibility of measuring, by real-time PCR, the levels of hsa-miR-18b-3p miRNA circulating in the serum of patients affected by head and neck cancer in an advanced stage (III and IV) (FIG. 6A) and, subsequently, of verifying a possible deregulation thereof before and after radiotherapy treatment (FIG. 6B). These first data not only demonstrated that the levels of circulating hsa-miR-18b-3p miRNA are easily measurable in biological fluids such as serum (FIG. 6A), but also demonstrate that the same correlate with the response to radiation-induced damage. Subsequently, the levels of hsa-miR-18b-3p miRNA were measured in the serum of patients enrolled in the framework of a project underway at the Istituto Nazionale Regina Elena, entitled “Identificazione di nuovi biomarcatori circolanti per la cachessia in pazienti affetti da tumors della testa e del collo trattati con radio-chemioterapia” (Identification of New Circulating Biomarkers for Cachexia in Patients Affected by Head and Neck Cancer Treated with Radiochemotherapy) and carried out in collaboration with the Operational Unit (UOC) of Radiotherapy, Otolaryngology and Head and Neck Surgery, the biological fluids bank and our departmental operational unit (UOSD) of oncogenomics and epigenetics. The study provides for the collection of serum from patients affected by head and neck cancer in an advanced stage (stages III and IV), before combined chemoradiotherapy treatment (baseline), at the end thereof (after treatment) and three months after the last cycle (follow up-FU) with the aim of detecting the presence of circulating biomarkers which identify the process of cachexia (FIG. 6C). The latter is a debilitating event which, like mucositis, is a direct consequence of antineoplastic treatment and can seriously compromise the quality of life of cancer patients. In three of the four patients analysed, an increase in the levels of circulating hsa-miR-18b-3p miRNA was revealed after the antineoplastic treatment and a subsequent decrease in the same in the sample taken at the follow-up (FIG. 60). The interpatient differences in expression levels might in some way be linked to the patient's susceptibility to developing oral mucositis and/or to the degree of severity thereof. This hypothesis could receive confirmation through the implementation of a targeted clinical study that examines the relationship between the levels of the 6 microRNAs and patient susceptibility, the development of mucositis and the degree of severity thereof. In this regard, a multicentre clinical study entitled: “Prospective Cohort Study on the Mutated TP53 Effect Modification on Chemotherapy-related Mucositis” is currently at the approval stage; it will entail collaboration between the Istituto Regina Elena, the Istituto San Gallicano, the University of Milano and the Princess Margaret Cancer Center.

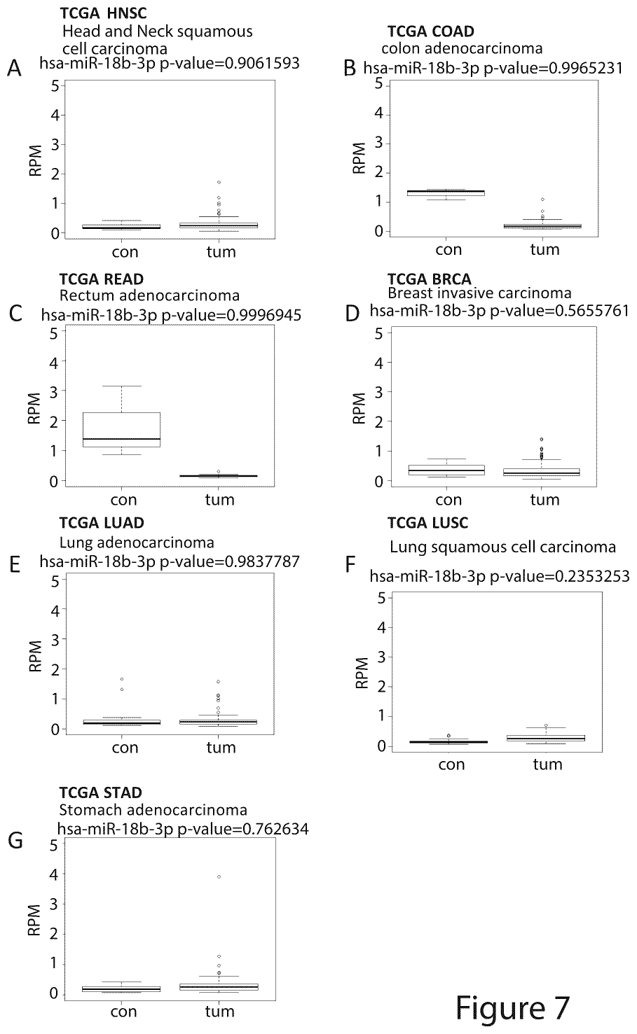

Through an interrogation of the TOGA public database it has been shown how miR-18b-3p levels are very low in tumour tissue and miR-18b-3p shows no difference in terms of expression between healthy tissue and tumour tissue in the different tumour tissues analysed, including: head and neck cancer (FIG. 7A), colon adenocarcinoma (FIG. 7B) and rectum adenocarcinoma (FIG. 70), breast carcinoma (FIG. 7D), lung adenocarcinoma (FIG. 7E) and lung squamous cell carcinoma (FIG. 7F) and gastric adenocarcinoma (FIG. 7G). This finding reinforces the idea of the specificity of miR-18b-3p as a biomarker of the mucositis inflammatory process irrespective of the type of tumour, but one that is specific for the damage of antitumour drugs on the oral mucosa.

The present invention has been described by an illustrative but not limitative way according to the preferred embodiments thereof, but it is to be understood that a person skilled in the art may introduce changes and/or modifications without going outside the scope of protection, as defined by the appended claims.

### REFERENCES

1. Vera-Lionch M, Oster G, Ford C M, Lu J, Sonis S: Oral mucositis and outcomes of allogeneic hematopoietic stem-cell transplantation in patients with hematologic malignancies. Support Care Cancer 2007, 15:491-496.
2. Trotti A, Belim L A, Epstein J B, Frame D, Fuchs H J, Gwede C K, Komaroff E, Nalysnyk L, Zilberberg M D: Mucositis incidence, severity and associated outcomes in patients with head and neck cancer receiving radiotherapy with or without chemotherapy: a systematic literature review. Radiother Oncol 2003, 66:253-262,
3. Elting L S, Cooksley C, Chambers M, Cantor S B, Manzulio E, Rubenstein E B: The burdens of cancer therapy. Clinical and economic outcomes of chemotherapy-induced mucositis, Cancer 2003, 98:1531-1539.
4. Elting L S, Cooksley C D, Chambers M S, Garden A S: Risk, outcomes, and costs of radiation-induced oral mucositis among patients with head-and-neck malignancies. Int J Radiat Oncol Biol Phys 2007, 68:1110-1120.
5. Sonis S T, Elting L S, Keefe D, Peterson D E, Schubert M, Hauer-Jensen M, Bekele B N, Raber-Durlacher J, Donnelly J P, Rubenstein E B: Perspectives on cancer therapy-induced mucosal injury: pathogenesis, measurement, epidemiology, and consequences for patients. Cancer 2004, 100:1995-2025.
6. Normando A G C, Rocha CL, de Toledo IP, de Souza Figueiredo P T, Dos Reis P E D, De Luca Canto G, Guerra E N S: Biomarkers in the assessment of oral mucositis in head and neck cancer patients: a systematic review and meta-analysis, Support Care Cancer 2017, 25:2969-2988.
7. Ramdas L, Giri U, Ashorn C L, Coombes K R, El-Naggar A, Ang K K, Story MD; miRNA expression profiles in head and neck squamous cell carcinoma and adjacent normal tissue. Head Neck 2009, 31:642-654.
8. Mitcheli P S, Parkin R K, Kroh E M, Fritz B R, Wyman S K, Pogosova-Agadjanyan E L, Peterson A, Noteboom J, O'Briant K C, Allen A, et al: Circulating microRNAs as stable blood-based markers for cancer detection. Proc Natl Acad Sci USA 2008, 105:10513-10518.
9. Wang J, Zhang K Y, Liu S M, Sen S: Tumor-associated circulating microRNAs as biomarkers of cancer. Molecules 2014, 19:1912-1938.
10. Chen X, Ba Y, Ma L, Cai X, Yin Y, Wang K, Guo J, Zhang Y, Chen J, Guo X, et al: Characterization of microRNAs in serum: a novel class of biomarkers for diagnosis of cancer and other diseases. Cell Res 2008, 18:997-1006.
11. Rapado-Gonzalez O, Majem B, Muinelo-Romay L, Alvarez-Castro A, Santamaria A, Gil-Moreno A, Lopez-Lopez R, Suarez-Cunqueiro MM: Human salivary microRNAs in Cancer. J Cancer 2018, 9:638-649.
12. Salazar-Ruales C, Arguello J V, Lopez-Cortes A, Cabrera-Andrade A, Garcia-Cardenas J M, Guevara-Ramirez P, Peralta P, Leone P E, Paz Y M C: Salivary MicroRNAs for Early Detection of Head and Neck Squamous Cell Carcinoma: A Case-Control Study in the High Altitude Mestizo Ecuadorian Population. Biomed Res Int 2018, 2018: 9792730.
13. Jingling T, Mengling F, Difan Z, Yiyang Ho, Jing Z, Hai L, Sherven S, Guanyu W and Qinghua D; miR-200c Modulates the Pathogenesis of Radiation-Induced Oral Mucositis. Oxidative Medicine and Cellular Longevity, vol. 2019, Article 10 2352079, 14 pages, 2019.
14. Elad S., Kin Fong Cheng K., Lalla R. V., Yarom N., Hong C., Logan R. M, Bowen J., Gibson R., Saunders D. P., Zadik Y., Ariyawardana A Correa M. E., Ranna V., and Bossi P. Cancer 2020, 126(19):4423-4431
15. Chou T C: Theoretical basis, experimental design, and computerized simulation of synergism and antagonism in drug combination studies. Pharmacol Rev 2006, 58:621-681.

1. A method for in vitro diagnosis and treatment of mucositis in a cancer patient undergoing antitumor therapy, comprising:
obtaining a measurement, in at least one biological sample of liquid biopsy taken before the antitumor therapy and in at least one biological sample of liquid biopsy taken after the antitumor therapy, the expression of an miRNA selected from the group consisting of hsa-miR-18b-3p, hsa-miR-494-3p, hsa-miR-1202, hsa-miR-3135b, hsa-miR-6769b-5p and hsa-miR-6875-5p;
identifying a change in the expression of said miRNAs in said at least one biological sample taken after the antitumor therapy compared to the expression of said miRNAs in said at least one biological sample taken before the antitumor therapy wherein a change indicates mucositis; and
carrying out at least one of (a), (b), or (c);
(a) administering a treatment indicated in guidelines provided by Multinational Association of Supportive Core in Cancer and International Society for Oral Oncology (MASCC/ISOO);
(b) increasing interval time before a subsequent antitumor therapy is administered; or
(c) varying dosage of the antitumor therapy.

2. The method according to claim 1, wherein the mucositis is at a pathogenetic stage of initiation of tissue damage.

3. The method according to claim 1, wherein one, more than one or all of the miRNAs comprise of hsa-miR-18b-3p.

4. The method according to claim 3, wherein a step of measuring the expression of one, more than one or all said miRNAs is carried out only in at least one biological sample taken after the antitumour therapy, when the one, more than one or all of the miRNAs consist of hsa-miR-18b-3p.

5. The method according to claim 1, wherein expression of more than one of the miRNAs is measured and wherein said more than one of the miRNAs are selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-5p, hsa-miR-18b-3p and hsa-miR-6875-5p, hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b, hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b, hsa-miR-18b-3p, hsa-miR-1202 and hsa-miR-6769b-5p; hsa-miR-18b-3p, hsa-miR-1202 and hsa-miR-6875-5p; hsa-miR-18b-3p, hsa-miR-3135b and hsa-miR-6769b-5p; hsa-miR-18b-3p, hsa-miR-3135b and hsa-miR-6875-5p; hsa-miR-18b-3p, hsa-miR-676911-5p and hsa-miR-6875-5p; hsa-miR-494-3p, hsa-miR-1202 and hsa-miR-3135b; miR-494-3p, hsa-miR-1202 and hsa-miR-6769b-5p; miR-494-3p, hsa-miR-1202 and hsa-miR-6875-5p; miR-494-3p, hsa-miR-3135b and hsa-miR-6769b-5p, miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b; hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, miR-3135b and hsa-miR-676911-5p; hsa-miR-18b-3p, miR-494-3p, miR-3135b and hsa-miR-6875-5p; hsa-miR-18b-3p, miR-494-3p, hsa-miR-6769b-5p and hsa-miR-6875-5p; hsa-miR-18b-3p, hsa-miR-1202, miR-3135b and hsa-miR-676911-5p; hsa-miR-18b-3p, hsa-miR-1202, miR-31350 and hsa-miR-6875-5p; hsa-miR-18b-3p, hsa-miR-1202, hsa-miR-6769b-5p and hsa-miR-6875-5p; hsa-miR-18b-3p, miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p, hsa-miR-494-3p, hsa-miR-1202, hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, hsa-miR-3135b and hsa-miR-6769b-5p; hsa-miR-18b-3p, hsa-miR-494-3p, hsa-miR-1202, hsa-miR-31.35b and hsa-miR-6875-5p; hsa-miR-18b-3p, hsa-miR-494-3p, hsa-miR-1202, hsa-miR-6769b-5p and hsa-miR-6875-5p; hsa-miR-181-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR-494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p.

6. The method according to claim 1, wherein the biological sample of liquid biopsy is selected from the group consisting of saliva, blood, plasma, serum, preferably saliva.

7. The method according to claim 1, wherein the antitumor therapy is selected from the group consisting of chemotherapy, radiotherapy and/or therapy with molecularly targeted drugs.

8. The method according to claim 7, wherein the chemotherapy or therapy with molecularly targeted drugs is selected from the group consisting of a PI3Kα inhibitor such as, for example, alpelisib, an alkylating agent such as, for example, cisplatin, oxaliplatin or ifosfamide, an antimetabolite such as, for example, 5-fluorouracile, capecitabin or azacytidine, an antimitotic such as, for example, paclitaxel, docetaxel or vinorelbine, an antitumour antibiotic such as, for example, doxorubicin, a topoisomerase I inhibitor such as, for example, irinotecan, ail EGFR inhibitor such as, for example, cetuximab, ail EGFR-TK inhibitor such as, for example, erlotinib, an mTOR inhibitor such as, for example, everolimus.

9. The method according to claim 1, wherein the method is carried out by Real-Time PCR, Microarray or RNA Next Generation Sequencing.

10. The method according to claim 1, wherein the antitumor therapy is against a tumor selected from the group consisting of solid tumours, such as, for example, head and neck cancer, or non-solid tumours, such as lymphoma or leukaemia.

11. The method according to claim 1, wherein the mucositis is a mucositis of the oropharyngeal cavity, such as, oral mucositis.

12-19. (canceled)

20. A kit for in vitro diagnosis of mucositis in a cancer patient undergoing antitumor therapy, said kit comprising:
one or more primer pairs and/or one or more probes complementary to more than one microRNA, said more than one microRNA being selected from the group consisting of hsa-miR-18b-3p and hsa-miR-494-3p; hsa-miR-18b-3p and hsa-miR-1202; hsa-miR-18b-3p and hsa-miR-3135b; hsa-miR-18b-3p and hsa-miR-6769b-5p; hsa-miR-18b-3p and hsa-miR-6875-5p; hsa-miR-494-3p and hsa-miR-1202; hsa-miR-494-3p and hsa-miR-3135b; hsa-miR-494-3p and hsa-miR-6769b-5p; hsa-miR-494-3p and hsa-miR-6875-5p; hsa-miR-1202 and hsa-miR-3135b; hsa-miR-1202 and hsa-miR-6769b-5p; hsa-miR-1202 and hsa-miR-6875-5p; hsa-miR-3135b and hsa-miR-6769b-5p; hsa-miR-3135b and hsa-miR-6875-5p; hsa-miR-6769b-5p and hsa-miR-6875-5p; hsa-miR-18b-3p, hsa-miR-494-3p and hsa-miR-1202; hsa-miR-18b-3p, hsa-miR-494-3p and hsa-miR-3135b; hsa-miR-18b-3p, hsa-miR-494-3p and hsa-miR-6769b-5p; hsa-miR-18b-3p, hsa-miR-494-3p and hsa-miR-6875-5p; hsa-miR-18b-3p, hsa-miR-1202 and hsa-miR-3135b; hsa-miR-18b-3p, hsa-miR-1202 and hsa-miR-6769b-5p; hsa-miR-18b-3p, hsa-miR-1202 and hsa-miR-6875-5p; hsa-miR-18b-3p, hsa-miR-3135b and hsa-miR-6769b-5p; hsa-miR-18b-3p, hsa-miR-3135b and hsa-miR-6875-5p; hsa-miR-18b-3p, hsa-miR-6769b-5p and hsa-miR-6875-5p; hsa-miR-494-3p, hsa-miR-1202 and hsa-miR-3135b; miR-494-3p, hsa-miR-1202 and hsa-miR-6769b-5p; miR-494-3p, hsa-miR-1202 and hsa-miR-6875-5p; miR-494-3p, hsa-miR-3135b and hsa-miR-6769b-5p; miR-494-3p, hsa-miR-3135b and hsa-miR-6875-5p; hsa-miR-494-3p, hsa-miR-6769b-5p and hsa-miR-6875-5p; hsa-miR-1202, hsa-miR-3135b and hsa-miR-6769b-5p; hsa-miR-1202, hsa-miR-3135b and hsa-miR-6875-5p; hsa-miR-1202, hsa-miR-6769b-5p and hsa-miR-6875-5p; hsa-miR-3135b, hsa-miR-6769b-5p and hsa-miR-6875-5p; hsa-miR-18b-3p, hsa-miR-494-3p, hsa-miR-1202 and hsa-miR-3135b: hsa-miR-18b-3p, hsa-miR-494-3p, hsa-miR-1202 and hsa-miR-6769b-5p; hsa-miR-18b-3p, hsa-miR-494-3p, hsa-miR-1202 and hsa-miR-6875-5p; hsa-miR-18b-3p, hsa-miR-494-3p, miR-3135b and hsa-miR-6769b-5p; hsa-miR-18b-3p, miR-494-3p, miR-3135b and hsa-miR-6875-5p; hsa-miR-18b-3p, miR-494-3p, hsa-miR-6769b-5p and hsa-miR-6875-5p; hsa-miR-18b-3p, hsa-miR-1202, miR-3135b and hsa-miR-6769b-5p; hsa-miR-18b-3p, hsa-miR-1202, miR-3135b and hsa-miR-6875-5p; hsa-miR-18b-3p, hsa-miR-1202, hsa-miR-6769b-5p and hsa-miR-6875-5p; hsa-miR-18b-3p, miR-3135b, hsa-miR-6769b-5p and hsa-miR-6875-5p; hsa-miR-494-3p, hsa-miR-1202, hsa-miR-3135b and hsa-miR-6769b-5p; hsa-miR-494-3p, hsa-miR-1202, hsa-miR-3135b and hsa-miR-6875-5p; hsa-miR-494-3p, hsa-miR-1202, hsa-miR-6769b-5p and hsa-miR-6875-5p; hsa-miR-494-3p, hsa-miR-3135b, hsa-miR-6769b-5p and hsa-miR-6875-5p; hsa-miR-1202, hsa-miR-3135b, hsa-miR-6769b-5p and hsa-miR-6875-5p; hsa-miR-18b-3p, hsa-miR-494-3p, hsa-miR-1202, hsa-miR-3135b and hsa-miR-6769b-5p; hsa-miR-18b-3p, hsa-miR-494-3p, hsa-miR-1202, hsa-miR-3135b and hsa-miR-6875-5 hsa-miR-18b-3p, hsa-miR-494-3p, hsa-miR-1202, hsa-miR-6769b-5p and hsa-miR-6875-5p; hsa-miR-18b-3p, hsa-miR-494-3p, hsa-miR-3135b, hsa-miR-6769b-5p and hsa-miR-6875-5p; hsa-miR-18b-3p, hsa-miR-1202, hsa-miR-3135b, hsa-miR-6769b-5p and hsa-miR-6875-5p; hsa-miR 494-3p, hsa-miR-1202, hsa-miR-3135b, hsa-miR-6769b-5p and hsa-miR-6875-5p; and hsa-miR-18b-3p, hsa-miR-494-3p, hsa-miR-1202, hsa-miR-3135b, hsa-miR-6769b-5p and hsa-miR-6875-5p, wherein said primers and/or probes include a detectable label;
and reagents for measuring the amount of said detectable label.

21. A method for treatment of a tumor in a cancer patient, comprising:
obtaining a measurement, in at least one biological sample of liquid biopsy from the cancer patient, the expression of an miRNA selected from the group consisting of hsa-miR-18b-3p, hsa-miR-494-3p, hsa-miR-1202, hsa-miR-3135b, hsa-miR-6769b-5p and hsa-miR-6875-5p;
subjecting the cancer patient to antitumor therapy selected from the group consisting of chemotherapy, radiotherapy and therapy with molecularly—targeted drugs;
obtaining a measurement, in at least one biological sample of liquid biopsy from the cancer patient after the antitumor therapy, the expression of the miRNA; and
determining whether a change has occurred in the expression of said miRNAs in said at least one biological sample taken after the antitumor therapy compared to the expression of said miRNAs in said at least one biological sample taken before the antitumor therapy.

22. The method of claim 21, wherein the antitumor therapy is selected from the group consisting of administering to the cancer patient a chemotherapy or drug selected from the group consisting of PI3Kα inhibitor, an alkylating agent, an antimetabolite, an antimitotic, an antitumour antibiotic, a topoisomerase I inhibitor, an EGFR inhibitor, an EGFR-TK inhibitor, and an mTOR inhibitor.

23. The method of claim 22, wherein the antitumor therapy is selected from the group consisting of administering a chemotherapy or drug selected from the group consisting of alpelisib, cisplatin, oxaliplatin, ifosfamide, 5-fluorouracile, capecitabin, azacytidine, paclitaxel, docetaxel, vinorelbine, doxorubicin, irinotecan, cetuximab, erlotinib, and everolimus.
